(19) United States
(12) Patent Application Publication
(10) Pub. No.: US 2011/0318397 A1
(43) Pub. Date: Dec. 29, 2011

(54) METHOD FOR PREPARING HIGH PURITY GINSENOSIDE RD USING LACTOBACILLUS CASEI AND COSMETIC COMPOSITION FOR ANTI-WRINKLE COMPRISING THE HIGH PURITY GINSENOSIDE RD

(75) Inventors: Seong Kweon Lee, Seoul (KR); Jueun Song, Seoul (KR); Young Dae Park, Seoul (KR); Sung-Hee Cho, Seoul (KR)

(73) Assignee: KOREA NATUAL SCIENCECO., LTD., Seoul (KR)

(21) Appl. No.: 13/114,904

(22) Filed: May 24, 2011

(30) Foreign Application Priority Data
May 24, 2010 (KR) 10-2010-0047949

(51) Int. Cl. A61K 8/63; C07J 17/00; A61Q 19/00; C12P 33/20

(52) U.S. Cl. 424/401; 435/53; 536/6; 514/26

(57) ABSTRACT

The present invention relates to a method for preparing high-purity ginsenoside Rd using Lactobacillus casei, more particularly to a method for preparing high-purity ginsenoside Rd using Lactobacillus casei and an anti-wrinkle cosmetic composition comprising the high-purity ginsenoside Rd as an active ingredient. Since the method of the present invention is simple and can economically prepare high-purity ginsenoside Rd extract, it may make the best use of anti-wrinkle effect of the ginsenoside Rd. In addition, the present invention can increase stability and decrease skin irritation by preparing water-soluble molecular capsule of high-purity ginsenoside Rd, thereby having advantage of increasing applicability ginsenoside Rd radically.

### TECHNICAL FIELD

The present invention relates to a method for preparing high-purity ginsenoside Rd using Lactobacillus casei, more particularly to a method for preparing high-purity ginsenoside Rd using Lactobacillus casei and an anti-wrinkle cosmetic composition comprising the high-purity ginsenoside Rd as an active ingredient.

### BACKGROUND

Ginseng (Panax ginseng CA Meyer) is a perennial dicotyledonous plant belonging to the family Araliaceae, the order Apiales. In the oriental medicine, it is known as the most important herb invigorating energy. Ginseng contains a lot of active components including saponins, essential oils, phytosterols, polyacetylenes, phenolic substances, polysaccharides, or the like, and is reported to have anti-diabetic, cardiovascular disorder improving, anti-arteriosclerotic, central nervous system controlling, brain function promoting, nerve cell protecting, anticancer, antioxidant, immune function promoting, sexual dysfunction improving, and dietary activities.

These activities of ginseng are mainly related to ginseng saponin. Currently, about 30 ginseng saponins are chemically identified. They are classified based on chemical structure into the protopanaxadiol (PPD) group (19 species), the protopanaxatriol (PPT) group (10 species) and the oleanane group (1 species). Ginseng has different saponin contents depending on species. Korean ginseng contains more kinds of saponins (ginsenosides) exhibiting pharmacological activities in more quantity than American ginseng cultivated in the US and Canada or Tienchi ginseng in China. Its unique components including Rh2, Rg3 and Rf ginsenosides make it the best of all ginsengs.

The skin consists of the epidermis, the dermis and the subcutaneous tissue. Consisting of various cell types, including keratin-producing keratinocytes, melanin-producing melanocytes, immune-related Langerhans cells, sense-related Merkel cells, or the like, the epidermis provides protection from external stimulus and pathogens, temperature control and maintenance of moisture and lipid constituents. The dermis consists of fiber components and matrix components. The fiber component collagen provides strength and tension to the skin and protects the skin. It accounts for 90% of the dermis. Elastin accounts for 3-4% of the dermis and provides elasticity. Collagen is synthesized by fibroblasts and is decomposed by collagenase and elastase. The matrix components include polymer substances such as hyaluronic acid having potent water-retaining ability, mucopolysaccharides, proteoglycans, etc. The dermis plays an important role in determining the physicochemical properties of the skin. It is closely related to skin aging since the blood capillaries and nerves distributed in the dermis provide nutrients to the epidermis.

In general, skin aging is known to be caused mainly by aging and external factors. As a person ages, the function and number of fibroblasts decline, resulting in reduced synthesis of fiber components (e.g., collagen and elastin), loss of water in the skin cells, and structural change in the horny layer. Also, the increased crosslinked collagen leads to reduction in skin softness, moist and elasticity. The external factors causing skin aging include pollution, wind, temperature, UV, etc. Particularly, UVB in the wavelength range of 290 to 320 nm is an important factor. By producing reactive oxygen species in the skin, UV results in oxidative damage to the lipids, proteins, nucleotides, enzymes, etc. that constitute the skin cells. As the oxidation products are accumulated in the body, they act as the cause of skin aging, inducing tissue damage, loss of constituents of the dermis, increased melanin production, cornification of the epidermis, or the like.

Accordingly, studies are carried out about cosmetic compositions capable of preventing skin damage caused by UV. Especially, retinol (vitamin A) and retinol derivatives (e.g., vitamin A palmitate) are studied extensively. However, since retinol is very unstable and is easily oxidized when exposed to the air, its effect does not last and there is a concern of skin irritation.

Accordingly, development of a cosmetic composition capable of significantly improving skin wrinkles and skin dryness and solving the skin irritation problem of the existing anti-wrinkle products is required.

Since ginseng or red ginseng exhibits various pharmacological effects and is known to be involved in anti-aging, it may be usefully used in cosmetics.

Korean Patent No. 10-0296430 discloses a cosmetic composition containing various ginsenoside saponins. However, the composition is directed to the treatment of hair rather than skin. Korean Patent Publication No. 10-2005-0088741 discloses a cosmetic composition for protecting skin containing various ginsenoside saponins, which contains ginsenoside Rg2 obtained by extracting ginseng as main component.

### SUMMARY

The inventors of the present invention have found out that, among the ginsenosides included in ginseng or red ginseng, ginsenoside Rd has an excellent effect in preventing and improving skin wrinkles, and have developed an anti-wrinkle cosmetic composition using the same.

The present invention is directed to providing a method for preparing high-purity ginsenoside Rd, including: (a) extracting saponins from ginseng or red ginseng; (b) fermenting the saponins by mixing them with Lactobacillus casei or a culture thereof; and (c) removing protopanaxatriol (PPT) ginsenosides.

The present invention is also directed to providing an anti-wrinkle cosmetic composition containing the high-purity ginsenoside Rd as an active ingredient.

The present invention is also directed to providing a composition for changing the composition of protopanaxadiol (PPD) ginsenosides, containing Lactobacillus casei or a culture thereof as an active ingredient.

In one general aspect, the present invention provides a method for preparing high-purity ginsenoside Rd, comprising: (a) extracting saponins from ginseng or red ginseng; (b) fermenting the saponins by mixing them with Lactobacillus casei or a culture thereof; and (c) removing PPT ginsenosides.

In another general aspect, the present invention provides a method for preparing high-purity ginsenoside Rd, comprising: (a) extracting saponins from ginseng or red ginseng; (b) fermenting the saponins by mixing them with Lactobacillus casei or a culture thereof; (c) removing PPT ginsenosides; and (d) molecularly encapsulating ginsenoside Rd with 2-hydroxypropyl-β-cyclodextrin (HPβCD).

In another general aspect, the present invention provides an anti-wrinkle cosmetic composition containing the high-purity ginsenoside Rd as an active ingredient.

### BRIEF DESCRIPTION OF THE DRAWINGS

FIG. 1 compares solubility of ginsenoside Rd and molecularly encapsulated ginsenoside Rd. FIG. 1 (A) shows that ginsenoside Rd is water insoluble, and FIG. 1 (B) shows that molecularly encapsulated ginsenoside Rd is water soluble and is completely dissolved in purified water.

FIG. 2 schematically shows molecularly encapsulated ginsenoside Rd.

FIG. 3 shows collagen synthesis by ginsenoside Rd.

FIG. 4 shows a cytotoxicity test result of ginsenoside Rd.

FIG. 5 shows increased stability of molecularly encapsulated ginsenoside Rd.

### DETAILED DESCRIPTION OF EMBODIMENTS

Hereinafter, exemplary embodiments will be described in detail with reference to the accompanying drawings.

The present invention provides a novel method for economically and effectively producing ginsenoside Rd, comprising:

(a) extracting saponins from ginseng or red ginseng;

(b) fermenting the extract obtained in the step (a) by mixing them with Lactobacillus casei or a culture thereof; and

(c) removing protopanaxatriol (PPT) ginsenosides from the fermentation product obtained in the step (b).

Ginsenoside Rd is a protopanaxadiol (PPD) ginsenoside compound having a structure represented by Chemical Formula 1. Ginsenoside Rd has not been used industrially since it is present in ginseng or red ginseng in trace amounts (less than 1%) and is poorly water-soluble. But, it is known to be highly soluble in polar solvents and be effective in arthritis, cartilage regeneration, collagen synthesis, or the like. Therefore, it is widely used in foods. However, its use for anti-wrinkle cosmetics has not been known yet.

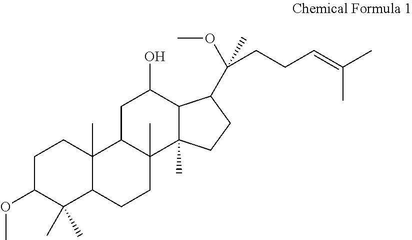

Hereinafter, the method for preparing high-purity ginsenoside Rd according to the present invention is described in detail.

In the Step (a), Saponins are Extracted from Ginseng or Red Ginseng.

The ginseng may be commonly used ginseng. Specifically, ginseng (Panax ginseng C. A. Meyer), American ginseng (Panax quinquefolium), Tienchi ginseng (Panax notoginseng), Japanese ginseng (Panax japonicum), dwarf ginseng (Panax trifolium) or Himalayan ginseng (Panax pseudoginseng) may be used. More specifically, ginseng may be used. The ginseng may be fresh ginseng or white ginseng.

Prior to extraction, the ginseng may be ground or pulverized to improve extraction efficiency. The grinding may be performed using a hand blender or a chopper. Specifically, a chopper may be used.

The extraction may be performed according to a method known in the art. For example, alcohol extraction, water extraction, organic solvent extraction, supercritical extraction, or the like may be employed, without being limited thereto. Specifically, water; a C₁-C₆ low alcohol such as methanol, ethanol, propanol, isopropanol, butanol, etc.; an organic solvent such as acetone, ether, benzene, chloroform, ethyl acetate, methylene chloride, hexane, hydrochloric acid, acetic acid, formic acid, citric acid, cyclohexane, petroleum ether, etc.; or a mixture thereof may be used.

The addition amount of the extraction solvent is not particularly limited. For example, the extraction solvent may be used in an amount of 1 to 20 times the dry weight of ginseng or red ginseng. In order to increase extraction efficiency, the extraction solvent may be used in an amount of 5 to 15 times the weight of ginseng or red ginseng, and the extraction may be repeated two or more times.

Extraction temperature may be 50 to 110° C., more specifically 90 to 100° C. Extraction time may be different depending on the extraction temperature and the extraction solvent. Typically, the extraction is carried out for 1 to 48 hours, specifically 2 to 6 hours. During the extraction, stirring may be performed using a shaker to further enhance the extraction efficiency.

In the Step (b), the Extract is Fermented by Mixing with Lactobacillus casei or a Culture Thereof.

The Lactobacillus casei used in the present invention may be one known in the art. Specifically Lactobacillus casei PM1 (KCCM 10766P) may be used. The Lactobacillus casei, specifically Lactobacillus casei PM1 (KCCM 10766P), is capable of converting the composition of PPD ginsenosides, i.e. decreasing the content of ginsenoside Rb1 or Rb2 and increasing the content of ginsenoside Rc or Rd, without affecting PPT ginsenosides during culturing/fermentation, which was confirmed by the inventors of the present invention for the first time.

In addition to the Lactobacillus casei itself, its culture may be used. As used herein, the culture refers to the Lactobacillus casei cultured for a predetermined period of time in a medium.

The culturing of the Lactobacillus casei or a culture thereof may be performed in large scale using the commonly employed lactic acid bacteria culturing method. A culture medium may contain carbon sources, nitrogen sources, vitamins and minerals. Specifically, a saponin-containing extract or crude extract may be added to a medium, or a culture of Lactobacillus casei may be added to a saponin-containing extract or crude extract. The culturing of the microorganism may be carried out under a common lactic acid bacteria culturing condition. For example, the culturing may be performed at 35° C. to 45° C., specifically 36° C. to 40° C., for 24 to 96 hours, specifically 48 to 96 hours, more specifically 72 hours. Most specifically, the culturing may be performed at 37° C. for about 72 hours.

In the Step (C), PPT Ginsenosides are Removed.

The removal of PPT ginsenosides may be performed by chromatography using a column known in the art. Specifically, after adsorbing ginsenoside in a column commonly used to remove sugars, e.g. a column packed with the synthetic filler HP-20, elution may be performed using, for example, absolute ethanol to completely remove the sugar components. Then, after crystallizing the ginsenosides, they may be recovered by adsorption and elution using a silica gel-packed column to obtain high-purity PPD ginsenosides.

The crystallization is required to more selectively separate the high-purity ginsenoside Rd. During the crystallization, constant rate may be maintained constant at −0.01 to −1° C./min. The weight ratio of the red ginseng fermentation product to ethanol may be 0.3-1:3-10, more specifically 0.5-0.7:5-7.

Since the high-purity ginsenoside Rd of the present invention has low water solubility and is easily oxidized, it may be encapsulated to improve stability and handlability. That is to say, the preparation method according to the present invention may further comprise a step (d) of molecularly encapsulating the ginsenoside Rd with 2-hydroxypropyl-β-cyclodextrin (HPCD).

HPCD is a β-cyclodextrin (β-CD) derivative prepared by attaching a hydroxypropyl group at the 2-glucose position of β-CD consisting of 7 glucose units bound together in a ring in order to enhance water solubility. It was developed recently for molecular encapsulation of lipophilic, water-insoluble active ingredients. Cyclodextrins (CDs) are widely used for molecular encapsulation. CDs consisting of 6 glucose units are called α-CD. Those consisting of 7, 8 and 12 to 15 glucose units are called, respectively, β-CD, 8 γ-CD and cluster CD. Different CDs are used depending on the molecular size of the active ingredient. They have suffered the water solubility problem or the stability problem, which could be solved through substitution of functional groups.

During the molecular encapsulation, the weight ratio of HPCD:high-purity ginsenoside Rd: water may be 1-10:0.5-5:1-10, more specifically 4-7:2-4:4-7.

The present invention further provides an anti-wrinkle cosmetic composition comprising the high-purity ginsenoside Rd prepared according to the present invention as an active ingredient. The cosmetic composition of the present invention may be prepared easily according to a method known in the art using the high-purity ginsenoside Rd prepared according to the present invention as well as one or more excipient and additive commonly used in the preparation of cosmetic compositions.

More specifically, the cosmetic composition of the present invention comprises the high-purity ginsenoside Rd prepared according to the present invention as an active ingredient and may be prepared into a base cosmetic composition (e.g., lotion, cream, essence, cleanser such as cleansing foam or cleansing water, pack, body oil, etc.), a coloring cosmetic composition (e.g., foundation, lipstick, mascara, makeup base, etc.), a hair product composition (e.g., shampoo, rinse, hair conditioner, hair gel, etc.), soap or the like, along with a dermatologically acceptable excipient. The excipient may include, for example, a skin emollient, a skin penetration enhancer, a coloring agent, an aromatic, an emulsifier, a thickener and a solvent, without being limited thereto. Also, a fragrance, a pigment, a sterilizer, an antioxidant, an antiseptic, a moisturizer, etc. may be further included, and a viscosity enhancer, minerals, synthetic polymer materials, etc. may be included to improve physical properties.

For example, the cosmetic composition of the present invention comprising the high-purity ginsenoside Rd prepared according to the present invention may be prepared easily into a facial cleanser or soap by adding the high-purity ginsenoside Rd to a commonly used facial cleanser or soap base. Also, a cream may be prepared by adding the high-purity ginsenoside Rd to a commonly used oil-in-water (O/W) type cream base. A fragrance, a chelating agent, a pigment, an antioxidant, an antiseptic, and synthetic or natural substances for improvement of physical properties such as proteins, minerals and vitamins may be further added.

The content of the high-purity ginsenoside Rd in the cosmetic composition of the present invention may be 0.0001 to 50 wt %, specifically 0.01 to 10 wt %, based on the total weight of the composition.

The Lactobacillus casei changes the composition of PPD ginsenosides during the fermentation. That is to say, it converts ginsenoside Rb1 or Rb2 to ginsenoside Rc or Rd (see Examples 1 and 2 and Table 1).

Accordingly, since Lactobacillus casei, specifically Lactobacillus casei PM1 (KCCM-10766P) decreases the content of ginsenoside Rb1 and greatly increases the content of ginsenoside Rd, it can be used to change the composition of the PPD ginsenosides.

### EXAMPLES

The examples and experiments will now be described.

The following examples and experiments are for illustrative purposes only and not intended to limit the scope of this disclosure.

### Example 1

### Fermentation of Red Ginseng (Ginseng)

100 g of dried red ginseng (ginseng) was added to 1 L 80% ethanol aqueous solution. After boiling at 100° C. for 2 hours under reflux followed by filtration, ethanol was evaporated from the resultant extract to finally obtain 22 g of a red ginseng extract. The result of analyzing the composition of the red ginseng extract is shown in Table 1.

To 1 L of a medium containing 22 g of the red ginseng extract, 100 g of a fermentation broth (a broth prepared by incubating Lactobacillus casei (KCCM 10766P) overnight in a medium containing 310.0 g of Proteose Peptone No. 3, 10.0 g of beef extract, 5.0 g of yeast extract, 20.0 g of dextrose, 1.0 g of Polysorbate 80, 2.0 g of ammonium citrate, 5.0 g of sodium acetate, 0.1 g of magnesium sulfate, 0.05 g of manganese sulfate and 20.0 g of dipotassium phosphate) was added. Then, fermentation was carried out for 72 hours at 37.5° C., while maintaining pH at 6.5.

The resulting fermentation solution was filtered through 0.2-μm filter to remove the bacteria and impurities. The filtrate was evaporated and the solid composition was analyzed. The result is shown in Table 1.

### Example 2

### Purification

The resulting fermentation solution was filtered through 0.2-μm filter to remove the bacteria and impurities. After adding the filtrate to a column packed with 200 g of the synthetic filler HP-20, the sugar components were completely removed by eluting with purified water. Then, the ginsenosides adsorbed to the synthetic filler HP-20 were recovered by eluting with absolute ethanol. After evaporating about half of ethanol from the recovered solution, the solution was cooled from 70° C. down to 5° C. at a constant rate of −0.2° C./min. After the cooling, crystals were recovered by filtration. The crystals, which are hardly soluble in water, were completely dissolved in a mixture solvent (chloroform:ethanol=6:4 based on weight). Then, the solution was added to a column packed with 100 g of silica gel and eluted with a mixture solvent (chloroform:ethanol=6:4 based on weight) to recover high-purity ginsenoside Rd. The composition of the active ingredients is shown in Table 1.

TABLE 1
Composition of ginsenosides in red ginseng (ginseng) extract,
fermentation product and purification product

| | Content (%) | | |
|---|---|---|---|
| | Concentrated extract | Fermentation | Purification |
| Ginsenosides | (ethanol) | product | product |
| Rg1 | 24.8 | 25.0 | 5.4 |
| Re | 14.5 | 13.8 | 2.3 |
| Rf | 5.1 | 5.0 | 1.5 |
| Rb1 | 31.3 | 2.0 | 3.1 |
| Rb2 | 16.0 | 9.8 | 12.6 |
| Rc | 4.9 | 13.7 | 17.8 |
| Rd | 3.3 | 31.4 | 57.2 |

* Result is after removal of solvent and water.

### Example 3

### Preparation of Molecularly Encapsulated Ginsenoside Rd

Since the crystals obtained in Example 2 are poorly water-soluble, they were molecularly encapsulated to prepare them into a stable, water-soluble form. 4 g of hydroxypropyl-β-cyclodextrin (HPβCD) was completely dissolved in 4 g of water at 60° C. to prepare a mixture solution. Then, 4 g of the high-purity ginsenoside Rd prepared in Example 2 was added to the water-HPβCD mixture solution and encapsulation was carried out by cooling to room temperature at a constant cooling rate of −0.2° C./min. Then, the product was kept at 4° C. for 7-10 hours in order to stabilize the capsules.

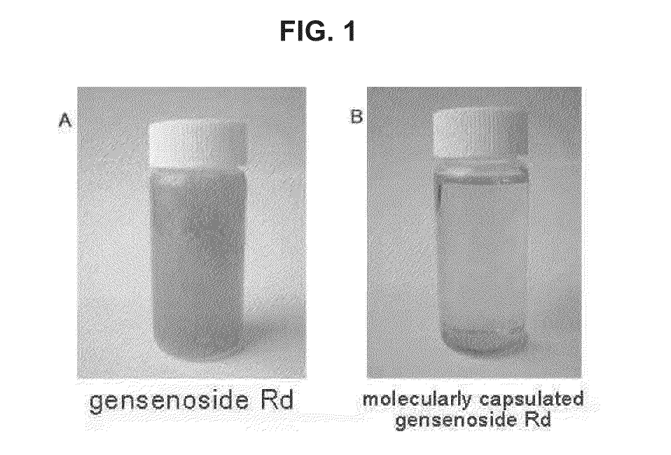

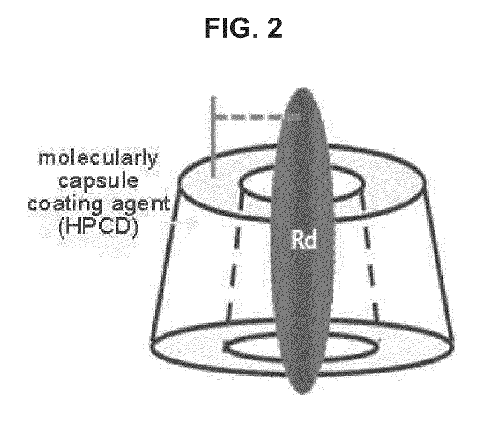

The prepared molecular capsules contained 3.25% of ginsenoside Rd. As seen from FIG. 1, the molecularly encapsulated ginsenoside Rd shows a significantly different solubility from that of ginsenoside Rd itself. FIG. 1 (A) shows that ginsenoside Rd is not completely dissolved in purified water, and FIG. 1 (B) shows that the molecularly encapsulated ginsenoside Rd is completely dissolved in purified water. FIG. 2 schematically shows the structure of the molecularly encapsulated ginsenoside Rd.

### Example 4

### Collagen Synthesis

Human fibroblasts (WI-38) that had been stored at −80° C. or in liquid nitrogen were used. The cells were uniformly placed on a 96-well plate, with 2.0×10⁴ cells/well.

After culturing in MEM (FBS 5%) for 24 hours, the medium was replaced with a fresh serum-free medium and the cells were treated with gradually diluted sample. The sample was the ginsenoside Rd prepared in Example 2, and commercially available standard ginsenoside Rg2 and standard retinol were used as control. After treatment with the sample, the cells were further cultured for 24 hours on a 96-well plate. Then, the quantity of collagen was measured using a collagen measurement kit (Procollagen type-I C-peptide MK101, Takara).

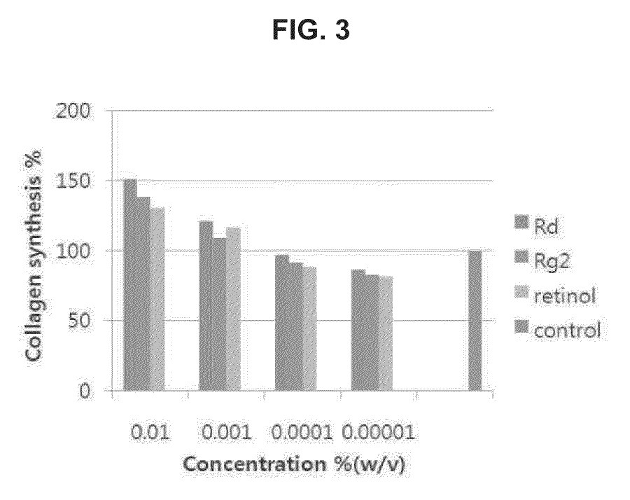

As seen from FIG. 3, ginsenoside Rd resulted in about 50% to 25% increased collagen production in the concentration range of 1×10⁻² to 1×10⁻³% (w/v), and slightly decreased collagen production in the low-concentration range of 1×10⁻⁴% (w/v) or lower, as compared to the control (sample-untreated cells). Rg2 showed about 40% to 10% increased collagen production in the concentration range of 1×10⁻² to 1×10⁻³% (w/v), and remarkably decreased collagen production in the concentration range of 1×10⁻⁴% (w/v) or lower, as compared to the control (sample-untreated cells). Retinol showed about 15 to 30% increased collagen production in the concentration range of 1×10⁻² to 1×10⁻³% (w/v), and maximum collagen production (30%) at the concentration 1×10⁻² (w/v) or lower, as compared to the control (sample-untreated cells). Accordingly, it can be seen that ginsenoside Rd exhibits very high collagen synthesis in the same concentration range, as compared to Rg2 or retinol.

### Example 5

### Stability of Molecularly Encapsulated Ginsenoside Rd

<5-1> Preparation of Cream

A nourishing cream containing the molecular capsule (3.25%; 0.33% of ginsenoside Rd) or ginsenoside Rd (0.33%) was prepared according to a commonly used method. 1 wt % of the molecular capsule prepared in Example 3 was mixed with 6 wt % of 1,3-butylene glycol, 4 wt % of glycerin, 5 wt % of liquid paraffin, 3 wt % of squalene, 1.5 wt % of Polysorbate 60, and purified water as balance to prepare the nourishing cream.

<5-2> Stability of Molecular Capsule

The nourishing creams prepared in Example <5-1> were kept at room temperature or at 45° C., and the change in color with time was observed.

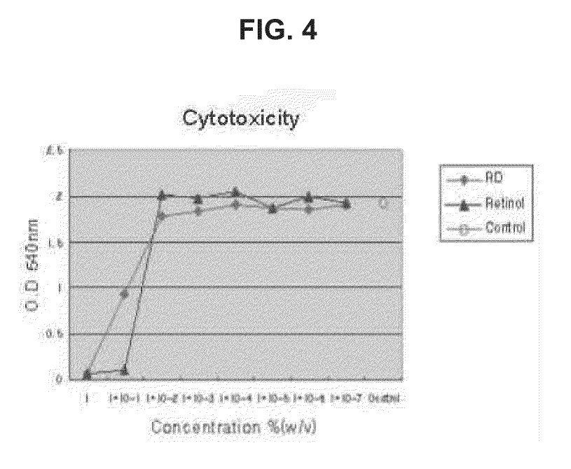

As seen from FIG. 4, after 60 days, the nourishing cream containing the molecularly encapsulated ginsenoside Rd showed less color change (browning) than the nourishing cream containing ginsenoside Rd itself.

### Example 6

### Cytotoxicity

MTT [3-(4,5-dimethylthiazol-2-yl)-2,5-diphenyltetrazolium bromide, Sigma M5655] assay was carried out to test the effect of the sample on proliferation of human fibroblasts.

The fibroblasts that had been stored at −80° C. or in liquid nitrogen were used. The cells were uniformly placed on a 96-well plate, with 2.0×10⁴ cells/well. After culturing in MEM (FBS 5%) for 24 hours, the medium was replaced with a fresh serum-free medium and the cells were treated with gradually diluted sample. The samples were ginsenoside Rd and retinol. After treatment with the sample, the cells were further cultured for 24 hours on a 96-well plate. Subsequently, after adding 10 μL of MTT solution (5 mg/mL) to each well, the cells were cultured for 4 hours. After removing the medium and adding 100 μL of dimethyl sulfoxide to each well followed by shaking for 10 minutes, absorbance was measured at 540 nm using an ELISA reader.

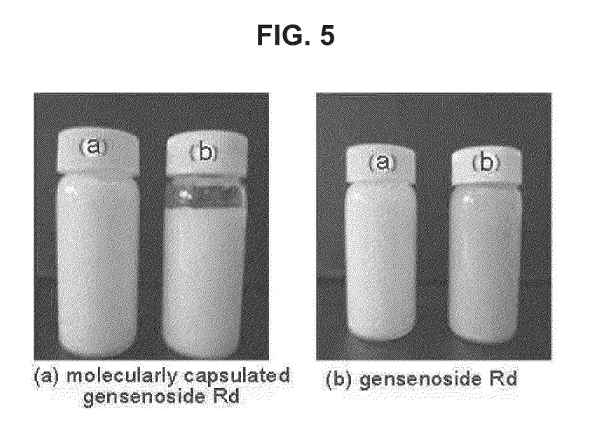

As seen from FIG. 5, cell viability was 100% (±10%) for both the samples ginsenoside Rd and retinol at concentrations below 1×10⁻¹% (w/v). Cytotoxicity was observed at higher concentrations. Thus, it can be seen that ginsenoside Rd exhibits no cytotoxicity at the high concentration of 1×10⁻¹% (w/v).

1. A method for preparing high-purity ginsenoside Rd, comprising the steps of:
(a) extracting saponins from ginseng or red ginseng;
(b) fermenting the extracts of (a) with Lactobacillus casei or a culture thereof; and
(c) removing PPT ginsenosides from fementant of (b).

2. The method of claim 1, wherein the ginseng is selected from the group consisting of ginseng (Panax ginseng C.A. Meyer), American ginseng (Panax quinquefolium), Tienchi ginseng (Panax notoginseng), Japanese ginseng (Panax japonicum), dwarf ginseng (Panax trifolium) or Himalayan ginseng (Panax pseudoginseng).

3. The method of claim 1, wherein the Lactobacillus casei is Lactobacillus casei PM1(KCCM 10766P).

4. The method of claim 1, wherein the extracting of step (a) is performed with a solvent selected from the group consisting of water, methanol, ethanol, propanol, isopropanol, butanol, acetone, ether, benzene, chloroform, ethyl acetate, methylene chloride, hexane, hydrochloric acid, acetic acid, formic acid, citric acid, cyclohexane, petroleum ether at 50 to 110° C. for 1 hr to 48 hrs.

5. The method of claim 1, wherein the fermenting of step (b) is performed at 35 to 45° C. for 24 to 96 hrs.

6. The method of claim 1, wherein the removing PPT ginsenosides of step (c) is performed by crystallization and chromatography.

7. The method of claim 1, wherein the method further comprises the step of (d) molecularly encapsulating the resultant of step (c) with HPCD.

8. An anti-wrinkle cosmetic composition comprising high-purity ginsenoside Rd prepared by the method of claim 1 or 7.
